(19) United States
(12) Patent Application Publication
(10) Pub. No.: US 2021/0346288 A1
(43) Pub. Date: Nov. 11, 2021

(54) PHARMACEUTICAL FORMULATION CONTAINING ACTIVE METABOLITES OF REMDESIVIR FOR INHALATION

(71) Applicants: Cai Gu Huang, Shrewsbury, MA (US); Ning He, Shanghai (CN)

(72) Inventors: Cai Gu Huang, Shrewsbury, MA (US); Ning He, Shanghai (CN)

(73) Assignee: Cai Gu Huang, Shrewsbury, MA (US)

(21) Appl. No.: 17/316,933

(22) Filed: May 11, 2021

Related U.S. Application Data
(60) Provisional application No. 63/023,222, filed on May 11, 2020.

(51) Int. Cl. A61K 9/00; A61K 31/706; A61K 9/08; A61K 47/02; A61K 47/12; A61K 47/18; A61K 47/40

(52) U.S. Cl. CPC A61K 9/0078; A61K 31/706; A61K 9/08; A61K 47/40; A61K 47/12; A61K 47/183; A61K 47/186; A61K 47/02

(57) ABSTRACT

The present invention relates to pharmaceutical formulations containing one or more active metabolites of remdesivir selected from alanine metabolite (Ala-met), nucleoside monophosphate, remdesivir monophosphate disodium salt and nucleoside triphosphate (NTP) or their pharmaceutically acceptable salts or solvates that are suitable for administration by soft mist inhalation or nebulization inhalation.

### PRIORITY STATEMENT

This application claims the benefit of the filing date of U.S. Provisional Patent Application No. 63/023,222, filed on May 11, 2020, which is incorporated herein by reference in its entirety.

### BACKGROUND OF THE INVENTION

The parent drug Remdesivir undergoes metabolization to form the active metabolites Alanine metabolite (Ala-met), Nucleoside monophosphate (Nuc), and Nucleoside Triphosphate (NTP). The chemical structures of each metabolite is given below:

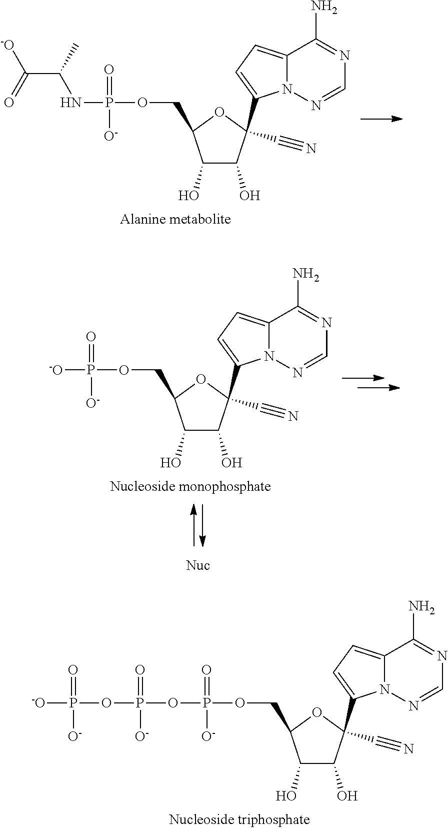

A 1-cyano-substituted adenine C-nucleoside ribose analogue (Nuc) exhibits antiviral activity against a number of RNA viruses. The mechanism of action of Nuc requires intracellular anabolism to the active triphosphate metabolite (NTP), which is expected to interfere with the activity of viral RNA-dependent RNA-polymerases (RdRp). Structurally, the 1-cyano group provides potency and selectivity towards viral RNA polymerases, but because of slow first phosphorylation kinetics, modification of parent nucleosides with monophosphate promoieties has the potential to greatly enhance intracellular NTP concentrations. The parent drug is a single Sp isomer of the 2-ethylbutyl 1-alaninate phosphoramidate prodrug, effectively bypasses the rate-limiting first phosphorylation step of the Nuc.

Remdesivir is a pro-drug of its parent adenosine analog, which is metabolized into an active nucleoside triphosphate (NTP) by the host and currently, an investigational broad-spectrum small-molecule antiviral drug that has demonstrated activity against RNA viruses in several families, including Coronaviridae (such as SARSCoV, MERS-CoV, and strains of bat coronaviruses capable of infecting human respiratory epithelial cells), Paramyxoviridae (such as Nipah virus, respiratory syncytial virus, and Hendra virus), and Filoviridae (such as Ebola virus).

As a nucleoside analog, Remdesivir acts as an RNA-dependent RNA polymerase, targeting the viral genome replication process. The RNA-dependent RNA polymerase is the protein complex that Coronavirus (CoVs) use to replicate their RNA-based genomes. After the host metabolizes Remdesivir into the active nucleoside triphosphate, the metabolite competes with adenosine triphosphate for incorporation into the nascent RNA strand. The incorporation of this substitute into the new strand results in premature termination of RNA synthesis, halting growth of the RNA strand after a few more nucleotides are added. Although CoVs have a proof-reading process that is able to detect and remove other nucleoside analogs, rendering them resistant to many of these nucleoside analogs, the active metabolites of Remdesivir seems to outpace this viral proof-reading activity, thus maintaining antiviral activity.

Remdesivir is currently administered intravenously, due to difficulties in administering it as an injectable solution. There are, however, side effects associated with intravenous administration of Remdesivir due to the long period of infusion time. An inhalation route of administration is a preferred administration route for delivery of drugs for the treatment of most of the respiratory diseases.

Surprisingly, we have found a new delivery method to more effectively and selectively deliver the active metabolites of remdesivir. This method advantageously improves deposition of the active metabolites of emdesiver in the lungs so that it can more effectively inhibit and remove virus from the lungs and other parts of the human body. This new delivery method involves soft mist inhalation or nebulization inhalation and presents clear and significant clinical benefits, such as improved availability at the target site, higher efficacy, and less side effects.

Furthermore, the delivery method, which involves administering a formulation by inhalation solution, has a significant advantages in that it achieves a better distribution of the active metabolites of remdesivir in the lung, which is beneficial when treating or curing a respiratory illness. Increased lung deposition of a drug delivered by inhalation is important for the treatment of virus infected diseases.

There is a significant need in the art to increase lung deposition when administering the active metabolites of remdesivir by inhalation. The soft mist or nebulization inhalation device disclosed in US20190030268 can significantly increase the lung deposition of inhalable drugs. These inhalers are suitable for therapeutic inhalation and can nebulize a small amount of a liquid formulation into an aerosol within a few seconds. These inhalers are particularly suitable for administering the liquid inhalation formulations of the invention.

Using a soft mist or nebulization device to administer the pharmaceutical formulations of the present invention allow an amount of less than about 70 microliters, preferably less than about 30 microliters, more preferably less than about 15 microliters, or even less of the pharmaceutical formulation to be nebulized in one puff, so that the inhalable part of aerosol corresponds to a therapeutically effective quantity. In one embodiment, the average particle size of the aerosol formed from one puff is less than about 15 microns. In one embodiment, the average particle size of the aerosol formed from one puff is less than about 10 microns.

Mesh based nebulization inhalation devices can also significantly increase the lung deposition of inhalable drugs and, thus, are also suitable for administering the active metabolites of remdesivir by inhalation.

### SUMMARY OF THE INVENTION

The present invention relates to pharmaceutical formulations containing one or more active metabolites of remdesivir (i.e., Alanine metabolite (Ala-met), Nucleoside monophosphate, and Nucleoside Triphosphate (NTP)) or their pharmaceutically acceptable salts or solvates that are suitable for administration by soft mist or nebulization inhalation. The pharmaceutical formulations according to the present invention meet high quality standards.

One aspect of the present invention is to provide a pharmaceutical formulation, containing one or more active metabolites of remdesivir or their pharmaceutically acceptable salts or solvates and, optionally, other inactive excipients that meets the high standards needed in so as to optimize nebulization of the formulation using a soft mist inhaler. Pharmaceutical stability of the formulations should be a storage time of some years, preferably at least one year, more preferably at least three years.

In one aspect, the formulation containing one or more active metabolites of remdesivir or their pharmaceutically acceptable salts or solvates and, optionally other inactive excipients is a solution. In one embodiment, the solution is nebulized using an inhaler device and the aerosol produced by the inhaler device falls reproducibly within a specified range.

In one aspect, the formulation containing one or more active metabolites of remdesivir or their pharmaceutically acceptable salts or solvates and, optionally, other inactive excipients can be administered by nebulization inhalation using an ultra-sonic based or an air pressure based nebulizer/inhaler. In one embodiment, the stability of active substances in the formulation is a storage time of a few months. In one embodiment the stability of the active substances in the formulation is a storage time of at least about 1 month. In one embodiment, the stability of active substances in the formulation is a storage time of at least about 6 months. In one embodiment the stability of active substances in the formulation is a storage time of at least about one year. In one embodiment the stability of active substances in the formulation is a storage time of at least about three years.

In one aspect, the formulation can be administered by soft mist inhalation using an atomizer inhaler. In one embodiment, the formulation exhibits long term stability. In one embodiment, the formulations has storage temperature is from about 1° C. to about 30° C. In one embodiment, the formulations storage temperature is from about 1° C. to about 30° C. In one embodiment, the formulations storage t temperature is from about 1° C. to about 30° C.

In one aspect, the formulation can be administered by nebulization inhalation using an ultrasonic jet or mesh nebulizer. The formulation has long-term stability. In one embodiment, the formulations storage temperature is from about 1° C. to about 30° C. In one embodiment, the formulations storage temperature is from about 1° C. to about 30° C. In one embodiment, the formulations storage temperature is from about 1° C. to about 30° C.

In one embodiment, the invention provides a method of treating a viral infection in a patient, wherein the viral is selected from the group consisting of Filoviridae (e.g., Ebola and Marburg virus), coronavirus, and COVID-19.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

FIG. 1 shows a longitudinal section through the atomizer in the stressed state.

FIG. 2 shows a counter element of the atomizer.

The use of identical or similar reference numerals in different figures denotes identical or similar features.

### DETAILED DESCRIPTION OF THE INVENTION

Administering an active substance by inhalation achieves a better distribution of active substances in the lungs. It is very important to increase lung deposition when an active substance is delivered by inhalation.

Therefore, there is a need in the art to improve drug delivery by inhalation so as to significantly increase lung deposition. The soft mist or nebulization inhalation device disclosed in US20190030268 can significantly increase the lung deposition of inhalable drugs. These inhalers can nebulize a small amount of a liquid formulation into an aerosol within a few seconds and are suitable for administering a therapeutic amount of the drug by inhalation. These inhalers are particularly suitable for this liquid formulation.

Using a soft mist or nebulization device to administer the pharmaceutical formulations of the present invention allow an amount of less than about 70 microliters, preferably less than about 30 microliters, more preferably less than about 15 microliters, or even less of the pharmaceutical formulation to be nebulized in one puff, so that the inhalable part of aerosol corresponds to a therapeutically effective quantity. In one embodiment, the average particle size of the aerosol formed from one puff is less than about 15 microns. In one embodiment, the average particle size of the aerosol is less than about 10 microns.

The nebulization devices used to administer the pharmaceutical formulations of the present invention are those in which an amount of less than about 8 milliliters, preferably less than about 2 milliliters, more preferably less than 1 milliliter, of the pharmaceutical formulation can be nebulized in one puff, so that the inhalable part of aerosol corresponds to a therapeutically effective quantity. In one embodiment, the average particle size of aerosol formed from one puff is less than 15 microns. In one embodiment, the average particle size of the aerosol formed from one puff is less than 10 microns.

A device of this kind for the propellant-free administration of a metered amount of a liquid pharmaceutical composition for inhalation is described in detail in, for example, US20190030268 entitled “inhalation atomizer comprising a blocking function and a counter”.

The pharmaceutical formulation in the nebulizer is converted into aerosol destined for the lungs. The pharmaceutical formulation is sprayed with the nebulizer by high pressure.

The pharmaceutical formulation is stored in a reservoir in this kind of inhalers. The formulations must not contain any ingredients which might interact with the inhaler to affect the pharmaceutical quality of the formulation or of the aerosol produced. In addition, the active substances in the pharmaceutical formulation exhibits good stability when stored and can be administered directly.

The pharmaceutical formulations of the invention for use with the inhaler described above preferably contain additives, such as the disodium salt of edetic acid (sodium edetate), to reduce the incidence of spray anomalies and to stabilize the formulation. Preferably, the formulations have a minimum concentration of sodium edetate.

In one aspect, the present invention provides a pharmaceutical formulation, which meets the high standards needed in order to be able to achieve optimal nebulization of a solution using a soft mist inhaler. The stability of the active substances in the formulation is preferably a storage time of some years. In one embodiment, the stability of the active substances in the formulation is at least one year. In one embodiment, the stability of the active substances in the formulation is at least three years.

In the formulations of the invention, the active substances are preferably selected from active metabolites of remdesivir and their pharmaceutically acceptable salts or solvates.

In the formulations of the invention, the active metabolites of Remdesivir or their pharmaceutically acceptable salts or solvates are preferably dissolved in a solvent. In one embodiment, the solvent is water.

In one aspect, the formulations is nebulized under pressure using an inhaler, which is preferably a soft mist inhaler, and the formulation is delivered by the aerosol produced, which falls reproducibly within a specified range.

In one aspect, the formulation comprising the active substance and, optionally, other inactive excipients is administered by nebulization inhalation. In one embodiment, the active substance has a mass median aerodynamic diameter of between about 1 micron and about 5 microns. This particle size advantageously is able to penetrate the lung on inhalation. In one embodiment, the invention provides a stable formulations containing the active pharmaceutical substance and, optionally, other excipients which can be administered by nebulization inhalation.

In the formulations according to the invention, the active substances or active ingredients is a remdesivir active metabolite. In one embodiment, the remdesivir active metabolite is selected from the group consisting of Alanine metabolite (Ala-met), Nucleoside monophosphate, and Nucleoside Triphosphate (NTP). In one embodiment, the active substance is dissolved in a solvent. In one embodiment, the solvent is selected from the group consisting of water, ethanol, and combinations thereof.

The current invention provides a method of treating a viral infection in a patient, wherein the viral is selected from Ebola and Marburg virus (Filoviridae); coronavirus, COVID-19, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichindé virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); influenza A virus (Orthomyxoviridae); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virustype 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Semliki Forest virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichindé virus, Tacaribe virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); human immunodeficiency virus type 1 (Retroviridae, Lentivirus); influenza AB virus (Orthomyxoviridae, the common ‘flu’ virus); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); hepatitis delta virus (hepatitis D virus); herpes simplex virus type 1, herpes simplex virus type 2 (Herpesviridae, Alphaherpesvirinae, Simplexvirus); Middle East Respiratory Syndrome (MERS) virus, severe acute respiratory syndrome CoV (SARS-CoV), human cytomegalovirus (Herpesviridae, Betaherpesvirinae, Cytomegalovirus).

The effective dose of the active pharmaceutical ingredient against COVID-19 depends on its bioavailability and clinical efficacy. In one embodiment, the effective dose of the active substance against COVID-19 is between about 1 mg and about 500 mg. In one embodiment, the effective dose of the active substance against COVID-19 is between about 10 mg and about 300 mg. In a preferred embodiment, the effective dose of the active substance against COVID-19 is between about 20 and about 100 mg. In a more preferred embodiment, the effective dose of the active substance against COVID-19 is between about 10 mg and about 30 mg.

The concentration of the active pharmaceutical ingredient in the finished pharmaceutical preparation depends on the desired therapeutic effect. In one embodiment, the concentration of the active substance in the soft mist formulation is between about 0.1 g/100 ml (1 mg/ml) and about 50 g/100 ml (500 mg/ml). In one embodiment, the concentration of the active pharmaceutical substance in the soft mist formulation is between about 1 g/100 ml (10 mg/ml) and 20 g/100 ml (200 mg/ml). In one embodiment, the concentration of the active substance in the soft mist formulation is between about 2 g/100 ml (20 mg/ml) and 20 g/100 ml (200 mg/ml).

The soft mist devices used to administer the pharmaceutical formulation of the invention can atomize about 10 to about 15 microliters of solution and atomize about 10 to about 15 times per use, so that the inhalable part of aerosol corresponds to a therapeutically effective quantity.

In one embodiment, an acid or a base can be added to the formulation as a pH adjusting agent to adjust the pH. In one embodiment, the formulation contains hydrochloric acid and/or a salt thereof.

Other comparable pH adjusting agents can be used in the present invention. Other pH adjusting agents include, but are not limited to, citric acid or/and sodium hydroxide.

Proper selection of the pH optimizes stability of active substances. In one embodiment, the pH ranges from about 2.0 to about 6.0. In a preferred embodiment, the pH ranges from about 3.0 to about 5.0. In a more preferred embodiment, the pH ranges from about 3.0 to about 4.0. In a further preferred embodiment, the pH ranges from about 3.0 to about 3.5.

In one embodiment, the formulations of the invention include edetic acid (EDTA) or one of the known salts thereof, disodium edetate or edetate disodium dihydrate, as a stabilizer or complexing agent. In one embodiment, the stabilizer is edetic acid and/or a salt thereof.

Other comparable stabilizers or complexing agents that can be included in the formulation include, but are not limited to, citric acid, edetate disodium, edetate disodium dihydrate.

The phrase complexing agent, as used herein, means a molecule which are capable of entering into complex bonds. Preferably, these compounds should have the effect of complexing cations. In one embodiment, the concentration of the stabilizers or complexing agents is from about 1 mg/100 ml to about 500 mg/100 ml. In one embodiment, the concentration of the stabilizers or complexing agents is from about 5 mg/100 ml to about 200 mg/100 ml. In one embodiment, the stabilizers or complexing agent is edetate disodium dihydrate in a concentration of about 10 mg/100 ml.

When the formulations are administered using an inhaler it is advantageous if all the ingredients of the formulation are present in solution.

The term additives, as used herein, means any pharmacologically acceptable and therapeutically useful substance which is not an active substance, but can be formulated together with the active substances in the pharmacologically suitable solvent, in order to improve the qualities of the formulation. Preferably, these substances have no pharmacological effects or no appreciable pharmacological effects or at least no undesirable pharmacological effects in the context of the desired therapy.

The additives include, but are not limited to, other stabilizers, complexing agents, antioxidants, surfactants, and/or preservatives which prolong the shelf life of the finished pharmaceutical formulation, vitamins, and other additives known in the art.

Suitable preservatives can be added to protect the formulation from contamination with pathogenic bacteria. Preservatives include, but are not limited to, benzalkonium chloride, benzoic acid, and sodium benzoate. In one embodiment, the formulations contain benzalkonium chloride as the only preservative. In one embodiment, the quantity of preservative ranges from about 2 mg/100 ml to about 300 mg/100 ml. In one embodiment, the preservative is benzalkonium chloride in an amount of about 10 mg/100 ml.

In one embodiment, the formulations of the invention include a solubility enhancing agent to aid the solubility of the active ingredient or other excipients. Suitable solubility enhancing agents include, but are not limited to, Tween 80 and cyclodextrin derivatives. In one embodiment, the solubility enhancing agent is a cyclodextrin derivative, or one of the known salts thereof. In one embodiment, the formulation contains sulfobutylether-β-cyclodextrin and/or a salt thereof.

In one embodiment, the solubility enhancing agent is selected from a group consisting of a surfactant and cyclodextrin. In one embodiment, the surfactant is selected from polysorbate, for example, polysorbate 20 and polysorbate 80; poloxamer; sodium dodecyl sulfate (SDS); sodium laurel sulfate; sodium octyl glycoside; polyethyl glycol; polypropyl glycol; copolymers, or any mixture thereof. In one embodiment, the solubility enhancing agent is a cyclodextrin selected from the group consisting of β-cyclodextrin, hydroxypropyl-cyclodextrin, sulfobutylether-β-cyclodextrin, and any combination thereof

Another aspect of the invention provides a stable pharmaceutical formulation which can be administered by soft mist inhalation using an atomizer inhaler. The formulation has long-term stability. In one embodiment, the formulations storage temperature is from about 1° C. to about 30° C. In one embodiment, the formulations storage temperature is from about 1° C. to about 30° C. In one embodiment, the formulations storage temperature is from about 1° C. to about 30° C. In one embodiment, the formulations storage t temperature of below 1° C. In one embodiment, the formulations storage temperature is from about 2° C. to about 8° C.

In one embodiment, the pharmaceutical formulations are administered by nebulization inhalation using a mesh based, an ultra-sonic based, or an air pressure-based nebulizer/inhaler. In one embodiment, the pharmaceutical formulation is a solution. In one embodiment, the formulations storage temperature is from about 1° C. to about 30° C. In one embodiment, the formulations have storage temperature is from about 1° C. to about 30° C. In one embodiment, the formulations storage temperature is from about 15° C. to about 30° C. In one embodiment, the formulations storage temperature of below 150° C. In one embodiment, the formulations storage t temperature is from about 2° C. to about 8° C.

A formulation for administration by nebulization typically contains the active ingredient and other excipients. In one embodiment, the mist droplets containing the active ingredient have a mass median aerodynamic diameter ranging from about 1 micron to about 10 microns. In one embodiment, the mist droplets containing the active ingredient have a mass median aerodynamic diameter ranging from about 1 microns to about 5 microns. This particle size is able to reach and be deposited in the lungs on inhalation.

In one embodiment, the formulations of the invention include sodium chloride. In one embodiment, the concentration of sodium chloride ranges from about 0 to about 0.9 g/100 ml.

In one embodiment, the concentration of the active substance in the formulation is between about 1 mg/100 ml and about 20 g/100 ml. In one embodiment, the concentration of the active substance is between about 5 mg/100 ml and about 1 g/100 ml.

In one embodiment, the formulations of the invention include a solubility enhancing agent. In one embodiment, the solubility enhancing agent is selected from a group consisting of a surfactant and a cyclodextrin. In one embodiment, the surfactant is selected from the group consisting of polysorbate, for example, polysorbate 20, polysorbate 80; poloxamer; tween-80; sodium dodecyl sulfate (SDS); sodium laurel sulfate; sodium octyl glycoside; polyethyl glycol; polypropyl glycol; copolymers, or any mixture thereof. In one embodiment, the solubilizing agent is a cyclodextrin selected from the group consisting of β-cyclodextrin, hydroxypropyl-cyclodextrin, sulfobutylether-β-cyclodextrin, and combinations thereof.

Another aspect of the invention provides a stable pharmaceutical formulation that can be administered using a mesh-based nebulization inhalation device. In one embodiment, the formulations exhibit long-term storage stability of substance. In one embodiment, the formulations have a storage time of at least about 6 months at a temperature of from about 15° C. to about 25° C. In one embodiment, the formulations have a storage time of at least about 12 months at a temperature of from about 15° C. to about 25° C. In one embodiment, the formulations have a storage time of at least about 24 months at a temperature of from about 15° C. to about 25° C. In one embodiment, the formulations storage temperature is below 15° C. In one embodiment, the formulations storage temperature is from about 2° C. to about 8° C.

Proper selection of pH provides optimal stability of the formulation and maintains the solubility of active substance. The pH can be adjusted to the desired pH by adding an acid, e.g., HCl, or by adding a base, e.g., NaOH or by a combination of HCl and NaOH to achieve the desired pH value. In one embodiment, a buffer is used to maintain the pH.

In one embodiment, the pH of the formulation ranges from about 3 to about 5.

In one embodiment, the formulations of the invention are filled into canisters to form a highly stable formulation for use in a nebulization device. The formulations exhibit substantially no particle growth or change of morphology. There is also no, or substantially no, problem of particles being deposited on the surface of either the canisters or the valves, so that the formulation can be discharged from the nebulization device with high dose uniformity. In one embodiment, the nebulizer is selected from the group consisting of an ultrasonic nebulizer, a jet nebulizer, and a mesh nebulizer. An example of a suitable nebulizer is a Pari eFlow nebulization inhaler.

In one embodiment, the inhalation device is a soft mist inhaler. In one embodiment, the pharmaceutical formulation is administered using an inhaler of the kind described herein. Here we should once again expressly mention the patent documents described hereinbefore, to which reference is hereby made.

A suitable device for administering a metered amount of a liquid pharmaceutical composition by soft mist inhalation is described in detail in, for example, US20190030268 entitled “inhalation atomizer comprising a blocking function and a counter”.

The pharmaceutical formulation is converted into an aerosol destined for the lungs by the nebulizer. The pharmaceutical solution is sprayed with the nebulizer by high pressure.

The inhalable device can be carried anywhere by the patient, since its cylindrical shape and handy size of less than 8 cm to 18 cm long, and 2.5 cm to 5 cm wide. The nebulizer sprays a defined volume of the pharmaceutical formulation out through small nozzles at high pressures, so as to produce inhalable aerosols.

The preferred atomizer comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, an inside part 17.

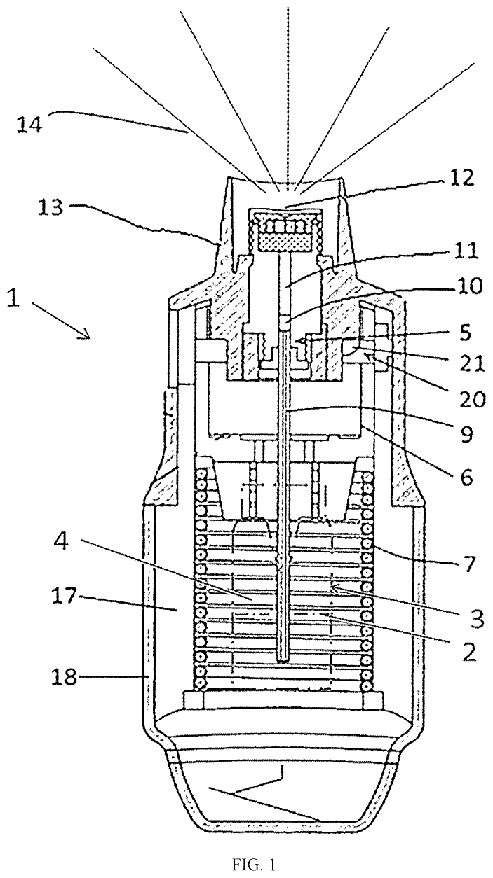

The inhalation atomizer 1 comprising the block function and the counter described above for spraying a medicament fluid 2 is depicted in the FIG. 1 in a stressed state. The atomizer 1 comprising the block function and the counter described above is preferably a portable inhaler and propellant-free.

FIG. 1 shows a longitudinal section through the atomizer in a stressed state.

For the typical atomizer 1 comprising the block function and the counter described above, an aerosol 14 that can be inhaled by a patient is generated through the atomization of the fluid 2, which is preferably formulated as a medicament liquid. The medicament is typically administered at least once a day, more specifically multiple times a day, preferably at predestined time gaps, according to how serious the illness affects the patient.

In an embodiment, the atomizer 1 described above has substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 described above is in the vessel 3 to provide, for example, up to 200 doses. A classical vessel 3 has a volume of about 2 to about 10 ml. A pressure generator 5 in the atomizer 1 is used to deliver and atomize the fluid 2, in a predestined dosage amount. Therefore, the fluid 2 could be released and sprayed in individual doses, specifically from 5 to 30 microliter.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 could be separated from the atomizer 1 for substitution.

In an embodiment, when drive spring 7 is stressed in the axial direction, the delivering tube 9, the vessel 3 along with the holder 6 will be shifted downwards. Then the fluid 2 will be sucked into the pressure room 11 through delivering tube 9 and the non-return valve 10.

In an embodiment, after releasing the holder 6, the stress is eased. During this process, the delivering tube 9 and closed non-return valve 10 are shifted back upward by releasing the drive spring 7. Consequently, the fluid 2 is under the pressure in the pressure room 11. Then the fluid 2 is pushed through the nozzle 12 and atomized into an aerosol 14 by the pressure. A patient could inhale the aerosol 14 through the mouthpiece 13, while the air is sucked into the mouthpiece 13 through air inlets 15.

The inhalation atomizer 1 comprising the block function and the counter described above has an upper shell 16 and an inside part 17, which can be rotated relative to the upper shell 16. A lower shell 18 is manually operable to attach onto the inside part 17. The lower shell 18 could be separated from the atomizer 1 so that the vessel 3 could be substituted and inserted.

In an embodiment, the inhalation atomizer 1 described above has the lower shell 18, which carries the inside part 17, being rotatable relative to the upper shell 16. As a result of rotation and engagement between the upper unit 17 and the holder 6, through a gear 20, the holder 6 axially moves the counter to the force of the drive spring 7 and the drive spring 7 is stressed.

In an embodiment, in the stressed state, the vessel 3 is shifted downwards and reaches a final position, which is demonstrated in the FIG. 1. The drive spring 7 is stressed under this final position. Then the holder 6 is clasped. Therefore, the vessel 3 and the delivering tube 9 are prevented from moving upwards so that the drive spring 7 is stopped from easing.

In an embodiment, the atomizing process occurs after releasing the holder 6. The vessel 3, the delivering tube 9 and the holder 6 are shifted back by the drive spring 7 to the beginning position. This is referred to herein as major shifting. While the major shifting occurs, the non-return valve 10 is closed and the fluid 2 is under the pressure in the pressure room 11 by the delivering tube 9, and then the fluid 2 is pushed out and atomized by the pressure.

In an embodiment, the inhalation atomizer 1 described above may preferably have a clamping function. During the clamping, the vessel 3 preferably performs a lifting shift for the withdrawal of the fluid 2 during the atomizing process. The gear 20 has sliding surfaces 21 on the upper shell 16 and/or on the holder 6, which could make holder 6 axially move when the holder 6 is rotated relative to the upper shell 16.

In an embodiment, the holder 6 is not blocked for too long and can carry on the major shifting. Therefore, the fluid 2 is pushed out and atomized.

In an embodiment, when the holder 6 is in the clamping position, the sliding surfaces 21 move out of engagement. Then the gear 20 releases the holder 6 for the opposite shift axially.

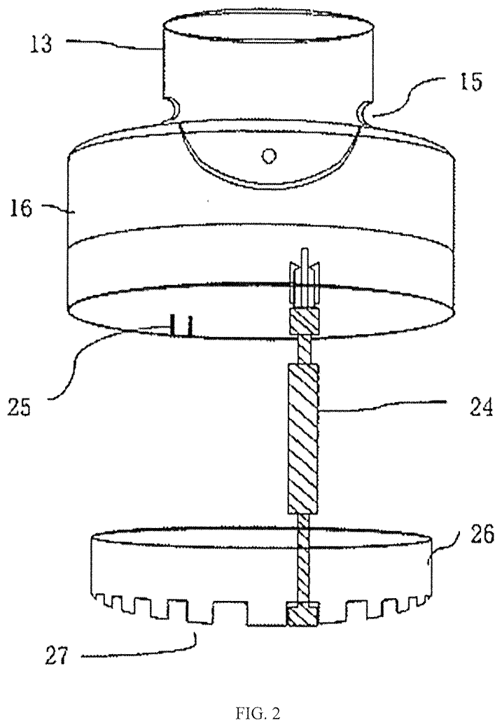

In an embodiment, the atomizer 1 preferably includes a counter element showed in FIG. 2. The counter element has a worm 24 and a counter ring 26. The counter ring 26 is typically circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in the counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter has a change. Once the predetermined number of actuations is achieved, Protrusion A and Protrusion B will encounter with each other and hence the counter will be prevented from further rotation. Therefore, the atomizer is blocked and stopped from further use. The number of actuations of the device can be counted by the counter.

Atomization devices include, but are not limited to, soft mist inhalers, ultrasonic atomizers, air compression atomizers, and mesh based atomizers.

The soft mist inhaler uses pressure to eject a metered dose solution of the active substance. Two high-speed jets are formed, and the two jets collide with each other to form droplets with smaller particles.

With an ultrasonic atomizer, the oscillation signal of the main circuit board is amplified by a high-power triode and transmitted to an ultrasonic wafer. The ultrasonic wafer converts electrical energy into ultrasonic energy. The ultrasonic energy atomizes the water-soluble active substance into tiny mist particles ranging in size from about 1 μm to about 5 μm at normal temperature. With the help of an internal fan, the medicine-containing particles are ejected.

An air compression atomizer is mainly composed of a compressed air source and an atomizer. The compressed gas is suddenly decompressed after passing through a narrow opening at high speed and a negative pressure is generated locally, so that the solution of the active substance is sucked out from a container because of a siphon effect. When subject to a high-speed air flow, the solution of the active substance is broken into small aerosol particles by collision.

Mesh based atomizers contain a stainless steel mesh covered with micropores having a diameter of about 3 μm. The number of micropores exceeds 1,000. The mesh is conical, with the cone bottom facing the liquid surface. Under the action of pressure, the vibration frequency of the mesh is about 130 KHz. The high vibration frequency breaks the surface tension of the drug solution contacted with the mesh and produces a low-speed aerosol.

### EXAMPLES

Materials and Reagents:
50% benzalkonium chloride aqueous solution purchased from Merck,
Edetate disodium dihydrate purchased from Merck,
Sodium hydroxide purchased from Titan reagents,
Hydrochloric acid purchased from Titan reagents,
Sodium Chloride purchased from Titan reagents,
Sulfobutylether-β-cyclodextrin purchased from Zhiyuan Biotechnology
Remdesivir monophosphate disodium salt is made by Anovent Pharmaceutical Co., Ltd.

### Example 1

Nebulization Inhalation Solution of Nucleoside Triphosphate (NTP)

The preparation of sample I, sample II, and sample III of a solution of Nucleoside Triphosphate for nebulization inhalation is as follows:

Sodium chloride and Nucleoside Triphosphate according to the contents in table 1, were added to 80 ml of purified water and the resulting mixture sonicated until completely dissolved. The resulting solution was then adjusted to the target pH with sodium hydroxide or hydrochloric acid. Finally, purified water was added to provide a final volume of 100 ml.

TABLE 1
Ingredient contents of sample I, sample II, sample III

| Ingredients | Sample I | Sample II | Sample III |
|---|---|---|---|
| Nucleoside | 5 g | 10 g | 2 g |
| Triphosphate | | | |
| Sodium chloride | 0.9 g | 0.6 g | 0.3 g |
| Hydrochloric acid or | To pH 3.0 | To pH 3.5 | To pH 4.0 |
| sodium hydroxide | | | |
| Purified water | Added | Added | Added |
| | to 100 ml | to 100 ml | to 100 ml |

### Example 2

Soft Mist Inhalation Solution of Nucleoside Triphosphate (NTP)

The preparation of sample IV, sample V, and sample VI of a solution of Nucleoside Triphosphate for soft mist inhalation is as follows:

Edetate Disodium Dihydrate, 50% benzalkonium chloride aqueous solution, and Nucleoside Triphosphate, according to the contents in table 2, were added to 80 ml of purified water and the resulting mixture sonicated until completely dissolved. The resulting solution was then adjusted to the target pH with sodium hydroxide or hydrochloric acid. Finally, purified water was added to provide a final volume of 100 ml.

TABLE 2
Ingredient contents of sample IV, sample V, sample VI

| Ingredients | Sample IV | | Sample V | | Sample VI | |
|---|---|---|---|---|---|---|
| Nucleoside | 10 | g | 15 | g | 20 | g |
| Triphosphate | | | | | | |
| Edetate Disodium | 10 | mg | 15 | mg | 20 | mg |
| Dihydrate | | | | | | |
| 50% benzalkonium | 20 | mg | 30 | mg | 40 | mg |
| chloride aqueous | | | | | | |
| solution | | | | | | |

| | | | |
|---|---|---|---|
| Hydrochloric acid or | To pH 3.0 | To pH 3.5 | To pH 4.0 |
| sodium hydroxide | | | |
| Purified water | Added | Added | Added |
| | to 100 ml | to 100 ml | to 100 ml |

### Example 3

Nebulization Inhalation Solution of Alanine Metabolite

The preparation of sample VII, sample VIII, and sample IX of a solution of Alanine metabolite for nebulization inhalation is as follows:

Sodium chloride and sulfobutylether-β-cyclodextrin, according to the contents in table 3, were dissolved in 80 ml of purified water. Alanine metabolite was added and the resulting mixture sonicated until completely dissolved. The resulting solution was then adjusted to the target pH with sodium hydroxide or hydrochloric acid. Finally, purified water was added to provide a final volume of 100 ml.

TABLE 3
Ingredient contents of sample VII, sample VIII, sample IX

| Ingredients | Sample VII | | Sample VIII | | Sample IX |
|---|---|---|---|---|---|
| Alanine metabolite | 500 | mg | 750 | mg | 1 g |
| Sulfobutylether-β- | 5 | g | 7.5 | g | 10 g |
| cyclodextrin | | | | | |
| Sodium chloride | 0.3 | g | 0.15 | g | 0 g |

| | | | |
|---|---|---|---|
| Hydrochloric acid or | To pH 3.0 | To pH 3.5 | To pH 4.0 |
| sodium hydroxide | | | |
| Purified water | Added | Added | Added |
| | to 100 ml | to 100 ml | to 100 ml |

### Example 4

Soft Mist Inhalation Solution of Alanine Metabolite

The preparation of sample X, sample XI, and sample XII of an Alanine metabolite solution for soft mist inhalation is as follows:

Sulfobutylether-β-cyclodextrin, edetate disodium dihydrate, and 50% benzalkonium chloride aqueous solution, according to the contents in table 4, were dissolved in 80 ml of purified water. Alanine metabolite was added and the resulting mixture sonicated until completely dissolved. The solution was then adjusted to the target pH with sodium hydroxide or hydrochloric acid. Finally, purified water was added to provide a final volume of 100 ml.

TABLE 4
Ingredient contents of sample X, sample XI, sample XII

| Ingredients | Sample X | | Sample XI | | Sample XII | |
|---|---|---|---|---|---|---|
| Alanine metabolite | 2 | g | 2.5 | g | 3 | g |
| Sulfobutylether-β- | 5 | g | 7.5 | g | 10 | g |
| cyclodextrin | | | | | | |
| Edetate Disodium | 10 | mg | 15 | mg | 20 | mg |
| Dihydrate | | | | | | |
| 50% benzalkonium | 20 | mg | 30 | mg | 40 | mg |

| | | | |
|---|---|---|---|
| chloride aqueous | | | |
| solution | | | |
| Hydrochloric acid or | To pH 3.0 | To pH 3.5 | To pH 4.0 |
| sodium hydroxide | | | |
| Purified water | Added | Added | Added |
| | to 100 ml | to 100 ml | to 100 ml |

### Example 5

Influence of Different pH on Stability:

Remdesivir monophosphate disodium salt is the salt of nucleoside monophosphate. The stability of remdesivir monophosphate disodium salt (referred to as RV-MP) is highly dependent on the pH. Four samples were prepared, having pH 3.0, 4.0, 5.0, 6.0. 23 ml of purified water was adjusted to the target pH with hydrochloric acid. RV-MP according to the amounts provided in table 5 was added to the solution, and the resulting mixture was sonicated until completely dissolved. The resulting mixture was adjusted to the target pH with hydrochloric acid. Finally, purified water was added to provide a final weight of 25.0 g.

The formulae of the 4 samples are shown in Table 5. The experimental results of influencing factors are shown in Tables 6-9.

TABLE 5
Formulation design of RV-MP compound
screening at different pH values

| Ingredients | Sample 1 | | Sample 2 | | Sample 3 | | Sample 4 | |
|---|---|---|---|---|---|---|---|---|
| RV-MP | 125 | mg | 125 | mg | 125 | mg | 125 | mg |
| Hydrochloric | Adjust | | Adjust | | Adjust | | Adjust | |
| acid | to pH | | to pH | | to pH | | to pH | |
| | 3.0 | | 4.0 | | 5.0 | | 6.0 | |
| Purified | 25.0 | g | 25.0 | g | 25.0 | g | 25.0 | g |

water

Impurities Detection Method: HPLC Detection Method
Column: YMC-triart C 18, 4.6*150 mm, S-5 μm, 12 nm;
Flow: 0.8 ml/min
Injection volume: 204,
Column temperature: 40° C.
A: Weigh 2.7 g of sodium dihydrogen phosphate, add 1 L of purified water, stir to dissolve, and filter to obtain.
B: Methanol

Gradient Elution:

| Time (min) | A % | B % |
|---|---|---|
| 0 | 95 | 5 |
| 12 | 95 | 5 |
| 13.5 | 5 | 95 |
| 15 | 95 | 5 |
| 20 | 95 | 5 |

The impurity results are as follows:

TABLE 6
The results of sample 1 (HCl pH 3.0)(conditions:
60° C. ± 2° C. / 75% ± 5% RH)

| | 0 day | 60° C. 5 day | Impurity |
|---|---|---|---|
| RRT | Impurities (%) | Impurities (%) | increased(%) |
| 0.330 | ND | 0.322 | 0.322 |
| 0.370 | 0.312 | 0.460 | 0.148 |
| 2.704 | ND | 9.063 | 9.063 |
| Maximum impurity | 0.312 | 9.063 | 8.751 |
| Total impurities | 0.312 | 9.845 | 9.533 |

ND: Not detected; same below

TABLE 7
The results of sample 2 (HCl pH 4.0) (conditions:
60° C. ± 2° C. / 75% ± 5% RH)

| | 0 day | 60° C. 5 day | Impurity |
|---|---|---|---|
| RRT | Impurities (%) | Impurities (%) | increased (%) |
| 0.330 | ND | 0.603 | 0.603 |
| 0.370 | 0.325 | 0.638 | 0.313 |
| 2.706 | ND | 9.294 | 9.294 |
| Maximum impurity | 0.325 | 9.294 | 8.969 |
| Total impurities | 0.325 | 10.535 | 10.21 |

TABLE 8
The results of sample 3 (HCl pH 5.0) (conditions:
60° C. ± 2° C. / 75% ± 5% RH)

| | 0 day | 60° C. 5 day | Impurity |
|---|---|---|---|
| RRT | Impurities (%) | Impurities (%) | increased (%) |
| 0.330 | ND | 2.509 | 2.509 |
| 0.370 | 0.311 | 1.900 | 1.589 |
| 2.700 | ND | 8.434 | 8.434 |
| Maximum impurity | 0.311 | 8.434 | 8.123 |
| Total impurities | 0.311 | 12.843 | 12.532 |

TABLE 9
The results of sample 4 (HCl pH 6.0) (conditions:
60° C. ± 2° C. / 75% ± 5% RH)

| | 0 day | 60° C. 5 day | Impurity |
|---|---|---|---|
| RRT | Impurities (%) | Impurities (%) | increased (%) |
| 0.328 | ND | 13.417 | 13.417 |
| 0.373 | 0.307 | 16.750 | 16.443 |
| 0.598 | 0.090 | 1.574 | 1.484 |
| 2.731 | 0.797 | 3.029 | 2.232 |
| Maximum impurity | 0.797 | 16.750 | 15.953 |
| Total impurities | 1.194 | 34.770 | 33.576 |

The above studies confirmed that the stability of RV-MP solution is highly dependent on the formulation pH. As can be seen from Tables 6-9, adjusting pH using HCl, the RV-MP formulations are stable at pH 3.0-4.0, with the highest stability at pH 3.0.

### Example 6

Adjust pH with Citric Acid:

The stability of remdesivir monophosphate disodium salt (referred to as RV-MP) is highly dependent on the pH. Five samples were prepared, having pH 3.0, 3.2, 3.5, 3.8, 4.0. 23 ml of purified water was adjusted to the target pH with citric acid. RV-MP according to the amounts provided in Table 10 was added to the solution, and the resulting mixture was sonicated until completely dissolved. The resulting mixture was adjusted to the target pH with citric acid. Finally, purified water was added to provide a final weight of 25.0 g.

The formulae of the 5 samples are shown in Table 10. The experimental results of influencing factors are shown in Tables 11-15.

TABLE 10
Formulation design of RV-MP compound screening at different pH values

| Ingredients | Sample 5 | Sample 6 | Sample 7 | Sample 8 | Sample 9 |
|---|---|---|---|---|---|
| RV-MP | 125 mg | 125 mg | 125 mg | 125 mg | 125 mg |
| 0.1M CA | Adjust | Adjust | Adjust | Adjust | Adjust |
| solution | to pH | to pH | to pH | to pH | to pH |
| | 3.0 | 3.2 | 3.5 | 3.8 | 4.0 |
| Purified | 25.0 g | 25.0 g | 25.0 g | 25.0 g | 25.0 g |
| water | | | | | |

TABLE 11
The results of sample 5 (CA pH 3.0) (conditions: 60° C./75% ± 5% RH)

| | 0 day impurities | 60° C. 4 days | Impurity increased |
|---|---|---|---|
| RRT | (%) | impurities (%) | (%) |
| 0.691 | ND | 0.583 | 0.583 |
| 1.254 | ND | 0.366 | 0.366 |
| 2.684 | 0.424 | 40.108 | 39.684 |
| Maximum impurity | 0.424 | 40.108 | 39.684 |
| Total impurities | 0.424 | 41.057 | 40.633 |

TABLE 12
The stability results of sample 6 (CA pH 3.2)
(conditions: 60° C. ± 2° C./75% ± 5% RH)

| | 0 day | 60° C. 4 day | Impurity increased |
|---|---|---|---|
| RRT | impurities (%) | impurities (%) | (%) |
| 2.688 | 0.123 | 5.885 | 5.762 |
| Maximum | 0.123 | 5.885 | 5.762 |
| impurity | | | |
| Total impurities | 0.123 | 5.885 | 5.762 |

TABLE 13
The stability results of sample 7 (CA pH 3.5)
(conditions: 60° C. ± 2° C./75% ± 5% RH)

| | 0 day | 60° C. 4 days | Impurity increased |
|---|---|---|---|
| RRT | Impurities (%) | Impurities (%) | (%) |
| 0.692 | ND | 0.286 | 0.286 |
| 2.687 | 0.171 | 4.830 | 4.659 |
| Maximum impurity | 0.171 | 4.830 | 4.659 |
| Total impurities | 0.171 | 5.116 | 4.945 |

TABLE 14
The stability results of sample 8 (CA pH 3.8)
(conditions: 60° C. ± 2° C./75% ± 5% RH)

| | 0 day | 60° C. 4 days | Impurity increased |
|---|---|---|---|
| RRT | Impurities (%) | Impurities (%) | (%) |
| 0.693 | ND | 0.315 | 0.315 |
| 2.685 | 0.225 | 5.122 | 4.897 |
| Maximum impurity | 0.225 | 5.122 | 4.897 |
| Total impurities | 0.225 | 5.437 | 5.212 |

TABLE 15
The stability results of sample 9 (CA pH 4.0)
(conditions: 60° C. ± 2° C./75% ± 5% RH)

| | 0 day | 60° C. 5 days | Impurity increased |
|---|---|---|---|
| RRT | impurities (%) | impurities (%) | (%) |
| 0.692 | ND | 0.318 | 0.318 |
| 2.685 | 0.365 | 5.356 | 4.991 |
| Maximum impurity | 0.365 | 5.356 | 4.991 |
| Total impurities | 0.365 | 5.674 | 5.309 |

Adjust pH with Hydrochloric Acid:

The stability of remdesivir monophosphate disodium salt (referred to as RV-MP) is highly dependent on the pH. Five samples were prepared, having pH 3.0, 3.2, 3.5, 3.8, 4.0. 23 ml of purified water was adjusted to the target pH with HCl. RV-MP according to the amounts provided in Table 16 was added to the solution, and the resulting mixture was sonicated until completely dissolved. Then the resulting mixture was adjusted to the target pH with HCl. Finally, purified water was added to provide a final weight of 25.0 g.

The formulae of the 5 samples are shown in Table 16. The experimental results of influencing factors are shown in Tables 17-21.

TABLE 16
Formulation design of RV-MP compound screening at different pH values

| Ingredients | Sample 10 | Sample 11 | Sample 12 | Sample 13 | Sample 14 |
|---|---|---|---|---|---|
| RV-MP | 125 mg | 125 mg | 125 mg | 125 mg | 125 mg |
| HCl | Adjust | Adjust | Adjust | Adjust | Adjust |
| | to pH | to pH | to pH | to pH | to pH |
| | 3.0 | 3.2 | 3.5 | 3.8 | 4.0 |
| Purified | 25.0 g | 25.0 g | 25.0 g | 25.0 g | 25.0 g |
| water | | | | | |

TABLE 17
The results of sample 10 (HCl pH 3.0)
(conditions: 60° C. ± 2° C./75% ± 5% RH)

| | 0 day Impurities | 60° C. 5 days | Impurity increased |
|---|---|---|---|
| RRT | (%) | Impurities (%) | (%) |
| 2.681 | 0.203 | 6.089 | 5.886 |
| Maximum impurity | 0.203 | 6.089 | 5.886 |
| Total impurities | 0.203 | 6.089 | 5.886 |

TABLE 18
The results of sample 11 (HCl pH 3.2)
(conditions: 60° C. ± 2° C./75% ± 5% RH)

| | 0 day Impurities | 60° C. 5 days | Impurity increased |
|---|---|---|---|
| RRT | (%) | Impurities (%) | (%) |
| 2.696 | ND | 4.790 | 4.790 |
| Maximum impurity | ND | 4.790 | 4.790 |
| Total impurities | ND | 4.790 | 4.790 |

TABLE 19
The results of sample 12 (HCl pH 3.5)
(conditions: 60° C. ± 2° C./75% ± 5% RH)

| | 0 day | 60° C. 5 days | Impurity increased |
|---|---|---|---|
| RRT | Impurities (%) | Impurities (%) | (%) |
| 2.696 | ND | 4.978 | 4.978 |
| Maximum impurity | ND | 4.978 | 4.978 |
| Total impurities | ND | 4.978 | 4.978 |

TABLE 20
The results of sample 13 (HCl pH 3.8) (conditions: 60° C. ± 2° C./
75% ± 5% RH)

| | 0 day | 60° C. 5 days | Impurity increased |
|---|---|---|---|
| RRT | Impurities(%) | Impurities (%) | (%) |
| 2.674 | 1.011 | 5.256 | 4.245 |
| Maximum impurity | 1.011 | 5.256 | 4.245 |
| Total impurities | 1.011 | 5.256 | 4.245 |

TABLE 21
The results of sample 14 (HCl pH 4.0)
(conditions: 60° C. ± 2° C./75% ± 5% RH)

| | 0 day | 60° C. 5 days | Impurity increased |
|---|---|---|---|
| RRT | Impurities(%) | Impurities (%) | (%) |
| 2.679 | 0.660 | 5.346 | 4.686 |
| Maximum impurity | 0.660 | 5.346 | 4.686 |
| Total impurities | 0.660 | 5.346 | 4.686 |

The above studies confirmed that the stability of RV-MP solution is highly dependent on the formulation pH and types of pH adjusters. As can be seen from above Tables, using citric acid to adjust the pH, the resulting solution is unstable, especially at pH3.0. Using HCl to adjust the pH, the resulting solution is more stable. The above studies confirmed that the RV-MP solution is unstable at 60° C. and needs to be stored at a low temperature. RV-MP solution needs to be stored at low temperature, such as at temperatures below about 15° C., for example at temperatures of about 2° C. to about 8° C.

### Example 7

Strong Degradation Test (Light and High Temperature Conditions)

Sample 15: 60 mg RV_MP dissolved in 100 ml purified water.

The API used in Example 7 is different from the batches of Examples 5 to 6, so the purity is different, and the difference in purity does not affect the light experiment.

The sample 15 was placed in light conditions, and the placement time was 2 hours, 4 hours, 6 hours, 24 hours, 48 hours, 72 hours, and the impurity detection method in Example 5 was used to detect the samples. The impurity results are shown in Table 22.

TABLE 22
Results of impurities degraded by strong light

| | light_2 H | light _4 H | light _6 H | light _24 H | light _48 H | light _72 H |
|---|---|---|---|---|---|---|
| | Impurities | Impurities | Impurities | Impurities | Impurities | Impurities |
| RRT | % | % | % | % | % | % |
| 0.371 | ND | ND | ND | 0.382 | 0.371 | 0.402 |
| 0.448 | ND | ND | ND | 0.614 | 0.580 | 0.611 |
| 0.594 | ND | ND | ND | 0.194 | 0.110 | 0.098 |
| 0.628 | ND | ND | ND | 0.544 | 0.505 | 0.511 |
| 2.709 | ND | ND | ND | 2.575 | 1.023 | 1.618 |
| Max impurities | ND | ND | ND | 2.575 | 1.023 | 1.618 |
| Total impurities | ND | ND | ND | 4.309 | 2.590 | 3.240 |

The impurity detection method of the present invention is the same as the impurity detection method in Example 5.

The above results prove that the RV-MP solution is unstable under light condition. Therefore, the storage of the sample needs to be keep away from light.

### Example 8

Aerodynamic Particle Size Distribution:

TABLE 23
Ingredient Contents of Sample 16 of 10 g Inhalation Solution Formulation
For Administration by Pari LC_Plus Inhalation

| Ingredients | Sample 16 |
|---|---|
| RV-MP | 50 mg |
| NaCl | 84 mg |
| target pH, using HCl adjust to target pH | Adjust to pH 3.2 |
| water | Add to 10 g |

Preparation Method of Sample 16:

9 ml of purified water was adjusted to the target pH with HCl. RV-MP according to the amount provided in Table 23 was added to the solution, and the resulting mixture was sonicated until completely dissolved. Then the resulting mixture was adjusted to the target pH with HCl. Finally, purified water was added to provide a final weight of 10 g.

The aerodynamic particle size distribution was determined using a Next Generation Impactor instrument (NGI). The inhaler used is Pari_e_flow. The Pari_e_flow inhaler was held close to the NGI inlet until no aerosol was visible. The flow rate of the NGI was set to 15 L/minute and was operated under ambient temperature and a relative humidity (RH) of 90±2%.

Sample 16 was discharged into the NGI. Fractions of the dose were deposited at different stages of the NGI, in accordance with the particle size of the fraction. Each fraction was washed from the stage and analyzed using HPLC. The results are provided in Table 24 below.

TABLE 24
Single Dose Level Distribution and Aerodynamic
Particle Size Distribution of RV-MP Inhalation
Formulation Sample 16 Administered by Pari _e_flow Inhalation

| | | Percentage | Cut-off |
|---|---|---|---|
| | | content | diameter |
| RV-MP Deposited | Dosage (mcg) | at all levels | (μm) |
| Throat | 72 | 0.84% | |
| Stage 1 | 180 | 2.11% | 14.10 |
| Stage 2 | 218 | 2.55% | 8.61 |
| Stage 3 | 1332 | 15.58% | 5.39 |
| Stage 4 | 3414 | 39.94% | 3.30 |
| Stage 5 | 2358 | 27.59% | 2.08 |
| Stage 6 | 782 | 9.15% | 1.36 |
| Stage 7 | 156 | 1.82% | 0.98 |
| Micro-Orifice Collector | 36 | 0.42% | |
| (MOC) | | | |
| Emitted dose (mcg) | 8548 | | |
| Fine particle dose (mcg) | 6746 | | |
| Fine Particle Fraction | 78.92% | | |
| (FPF) | | | |

Fine Particle Fraction (FPF) is the proportion of fine particle dose in the released dose.

$$F P F = \frac{Mass < 5 μm}{Mass Total dose} .$$

The larger the FPF value, the higher the atomization efficiency.

### Example 9

Nebulization Inhalation Solution of Remdesivir Monophosphate Disodium Salt.

The preparation of sample 17, and sample 18 of a solution of remdesivir monophosphate disodium salt for nebulization inhalation is as follows:

9 ml of purified water was adjusted to the target pH with HCl. Remdesivir monophosphate disodium salt according to the amounts provided in Table 25 was added to the solution, and the resulting mixture was sonicated until completely dissolved. Then the resulting mixture was adjusted to the target pH with HCl. Finally, purified water was added to provide a final weight of 10 g.

TABLE 25
Ingredient Contents of Sample 17 and Sample 18 of
10 g Inhalation Solution Formulation

| Ingredients | Sample 17 | Sample 18 |
|---|---|---|
| RV-MP | 100 mg | 200 mg |
| NaCl | 84 mg | 84 mg |
| target pH, using HCl adjust to | Adjust to pH 3.0 | Adjust to pH 3.5 |
| target pH | | |
| water | Add to 10 g | Add to 10 g |

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

1. A liquid, propellant-free pharmaceutical formulation comprising: (a) one or more active metabolites of remdesivir; and (b) a solvent.

2. The pharmaceutical formulation of claim 1, wherein one or more active metabolites of remdesivir are selected from the group consisting of alanine metabolite, nucleoside monophosphate, remdesivir monophosphate disodium salt, nucleoside triphosphate, and combinations thereof.

3. The pharmaceutical formulation of claim 1, wherein active metabolites of remdesivir are present in an amount ranging from about 0.1 g/100 ml to about 50 g/100 ml.

4. The pharmaceutical formulation of claim 1, wherein the one or more active metabolites of remdesivir are present in an amount ranging from about 10 mg/100 ml to about 20 g/100 ml.

5. The pharmaceutical formulation of claim 1, further comprising a preservative selected from the group consisting of benzalkonium chloride, benzoic acid, sodium benzoate, and combinations thereof.

6. The pharmaceutical formulation of claim 4, wherein the preservative is present in an amount ranging from about 2 mg/100 ml to about 300 mg/100 ml.

7. The pharmaceutical formulation of claim 1, wherein the solvent is water.

8. The pharmaceutical formulation of claim 1, further comprising a stabilizer selected from the group consisting of tween-80, poloxamer, polyoxyethylated castor oil, polyethylene glycol, solutol HS 15, polyvinylpyrrolidone, a cyclodextrin derivative, sulfobutylether β-cyclodextrin, and combinations thereof.

9. The pharmaceutical formulation of claim 8, wherein the stabilizer is present in an amount ranging from about 1 mg/100 ml to about 500 mg/100 ml.

10. The pharmaceutical formulation of claim 1, further comprising a pH adjuster selected from the group consisting of hydrochloric acid or citric acid, and wherein the pharmaceutical formulation has a pH ranging from about 2.0 to about 4.0.

11. The pharmaceutical formulation of claim 1, further comprising hydrochloric acid, and wherein the pharmaceutical formulation has a pH ranging from about 3.0 to about 3.5.

12. The pharmaceutical formulation of claim 1, further comprising an osmotic pressure regulator.

13. The pharmaceutical formulation of claim 1, further comprising sodium chloride.

14. The pharmaceutical formulation of claim 1, wherein the storage temperature of the formulation is below about 15° C.

15. The pharmaceutical formulation of claim 1, wherein the formulation is stored under dark conditions.

16. A method for administering the pharmaceutical formulation of claim 1, comprising nebulizing a defined amount of the pharmaceutical formulation by applying pressure to the pharmaceutical formulation to force the pharmaceutical formulation through a nozzle to provide an inhalable aerosol.

17. The method according to claim 16, wherein the defined amount of the pharmaceutical formulation ranges from about 5 microliters to about 30 microliters.

18. The pharmaceutical preparation according to claim 16, wherein aerosol has a D50 of less than about 10 μm.

19. A method for administering the pharmaceutical formulation of claim 1, comprising nebulizing the pharmaceutical formulation using an soft mist inhaler.

20. The method according to claim 19, wherein the soft mist inhaler comprises a block function and a counter.

21. A method for administering to the pharmaceutical formulation of claim 1, comprising nebulizing the pharmaceutical formulation with an inhaler selected from the group consisting of a soft mist inhaler, an ultrasonic atomizer, an air compression atomizer, and a mesh based atomizer.

22. A method of treating a virus infection in a patient, comprising administering to the patient the pharmaceutical formulation of claim 1.

23. The method according to claim 22, wherein the virus is selected from an Ebola and Marburg virus (Filoviridae); coronavirus, new coronavirus COVID-19, Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichindé virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Semliki Forest virus, O'nyong-nyong virus, rubella (German measles) virus (Togaviridae, Rubivirus); rabies virus, Lagos bat virus, Mokola virus (Rhabdoviridae, Lyssavirus); Guanarito virus, Sabia virus, Lassa virus (Arenaviridae, Mammarenavirus); Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); influenza AB virus (Orthomyxoviridae, the common ‘flu’ virus); Hendra virus, Nipah virus (Paramyxoviridae, Paramyxovirinae, Henipavirus); measles virus (Paramyxoviridae, Paramyxovirinae, Morbillivirus); variola major (smallpox) virus (Poxviridae, Chordopoxvirinae, Orthopoxvirus); human hepatitis B virus (Hepadnaviridae, Orthohepadnavirus); Middle East Respiratory Syndrome (MERS) virus, severe acute respiratory syndrome CoV (SARS-CoV), Marburg virus, and hepatitis delta virus (hepatitis D virus).

24. The method according to claim 23, wherein the effective dose of the active substance against COVID-19 is between about 10 mg and about 300 mg.

25. The method according to claim 23, wherein the effective dose of the active substance against COVID-19 is between about 10 mg and about 30 mg.

26. The pharmaceutical formulation of claim 1 comprising:
(i) nucleoside triphosphate in an amount ranging from 10 g/100 mL to about 20 g/100 mL,
(ii) edetate disodium dihydrate in an amount of about 1 mg per 1 g of nucleoside triphosphate,
(iii) 50% benzalkonium chloride aqueous solution in an amount of about 2 mg per 1 g of nucleoside triphosphate, and
(iv) water
wherein the formulation is an aqueous solution, and
wherein the pH of the formulation ranges from about 3.0 to about 4.0.

27. The pharmaceutical formulation of claim 1 comprising:
(i) alanine metabolite in an amount ranging from 500 mg/100 mL to about 1 g/100 mL,
(ii) sulfobutylether-β-cyclodextrin in an amount of about 10 g mg per 1 g of alanine metabolite,
(iii) sodium chloride in an amount ranging from about 0 g/100 mL to about 0.3 g/100 mL, and
(iv) water
wherein the formulation is an aqueous solution, and
wherein the pH ranges from about 3.0 to about 4.0.

28. The pharmaceutical formulation of claim 1 comprising:
(i) alanine metabolite in an amount ranging from 2 g/100 mL to about 3 g/100 mL,
(ii) sulfobutylether-β-cyclodextrin in an amount of about 2.5 mg to about 3.3 mg per 1 g of alanine metabolite,
(iii) edetate disodium dihydrate in an amount of about 5 mg to about 6.6 mg per 1 g of alanine metabolite,
(iv) 50% benzalkonium chloride aqueous solution in an amount of about 10 mg to about 13.3 mg per 1 g of alanine metabolite, and
(iv) water
wherein the formulation is an aqueous solution, and
wherein the pH ranges from about 3.0 to about 4.0.

29. The pharmaceutical formulation of claim 1 comprising:
(i) remdesivir monophosphate disodium salt in an amount ranging from 0.1 g/100 mL to about 50 g/100 mL,
(ii) NaCl in an amount of about 0 g/100 ml to about 0.9 g/100 ml,
(iii) edetate disodium dihydrate in an amount of about 0 mg to about 6.6 mg per 1 g of remdesivir monophosphate disodium salt,
(iv) 50% benzalkonium chloride aqueous solution in an amount of about 0 mg to about 13.3 mg per 1 g of remdesivir monophosphate disodium salt, and
(v) water
wherein the formulation is an aqueous solution, and
wherein the pH ranges from about 3.0 to about 3.5.
